(19) United States
(12) Patent Application Publication
(10) Pub. No.: US 2019/0200587 A1
(43) Pub. Date: Jul. 4, 2019

(54) SINGLE DOSE RECOMBINANT BOVINE FSH AFTER FOLLICULAR SYNCHRONISATION

(71) Applicant: CEVA SANTE ANIMALE, LIBOURNE (FR)

(72) Inventors: ALEXANDRE SOUZA, CARIGNAN DE BORDEAUX (FR); NAOMI ISAKA, LIBOURNE (FR); AUDE CARRIE, LES BILLAUX (FR); DOMINIQUE THIBAUD, BORDEAUX (FR)

(21) Appl. No.: 16/360,285

(22) Filed: Mar. 21, 2019

Related U.S. Application Data
(63) Continuation of application No. 14/443,193, filed on May 15, 2015.

(30) Foreign Application Priority Data
Nov. 16, 2012 (EP) 12306428.9

(51) Int. Cl. A01K 67/02; A61K 38/24; A61K 31/557; A61K 31/56; A61K 45/06

(52) U.S. Cl. CPC A01K 67/02; A61K 38/24; A61K 45/06; A61K 31/56; A61K 31/557

(57) ABSTRACT

The present invention relates to methods and compositions for increasing reproduction performance in non-human mammals using a biologically active recombinant Follicle Stimulating Hormone (rFSH). The invention also relates to methods for increasing ovulation, embryo production or pregnancies in non-human mammals using rFSH. The invention may be used in particular in ungulates such as bovines or equines.

### CROSS-REFERENCE TO RELATED APPLICATIONS

This application is a continuation of U.S. Ser. No. 14/443,193, filed May 15, 2015, which is the U.S. national stage application of International Patent Application No. PCT/EP2013/073928, filed Nov. 15, 2013.

The Sequence Listing for this application is labeled “Seq-List.txt” which was created on Jul. 28, 2015 and is 7 KB. The entire contents of the sequence listing is incorporated herein by reference in its entirety.

The present invention relates to methods and compositions for increasing reproduction performance in non-human mammals using a biologically active recombinant Follicle Stimulating Hormone (rFSH). The invention also relates to methods for increasing ovulation, embryo production, or pregnancies in non-human mammals using rFSH. The invention may be used in particular in ungulates such as bovines or equines, in artificial insemination or in vitro fertilization programs.

### BACKGROUND OF THE INVENTION

The ability to increase reproductive performance in non-human mammals such as cattle, horses or other ungulates can have a significant benefit to owners. This can be achieved through increasing fertility and/or fecundity in such non-human mammals. Currently, several methods or protocols are proposed to increase reproductive performance, based on the use of estradiol and other hormones such as GnRH (Gonadotropin Releasing Hormone), LH (Luteinizing Hormone), FSH (Follicle Stimulating Hormone), or progestagens (such as progesterone) to mimic natural or close to natural levels of hormones occurring at the target specie. Some of these protocols include a step to synchronize ovulation in females to facilitate stimulation, growth and ovulation of follicles in a synchronized fashion, allowing fixed-time artificial insemination and avoiding the necessity of estrus detection. These so-called synchronization protocols are widely used in commercial dairy and beef herds worldwide.

One of the most common treatments used to synchronize the emergence of follicular waves involves the use of different types of estrogens. However, this steroid hormone cannot be used in many parts of the world and alternative methods have been developed to control follicular and luteal phases, aiming for instance for the improvement of the superovulation process in embryo-donor cows in countries where estradiol is not available. An approach that seems to be a promising option is to initiate FSH treatments at the time of the emergence of a new follicular wave, following a GnRH-induced ovulation. In addition, because the half-lives of currently available FSH products are generally short (approximately 6 h), superovulatory protocols involve twice a day FSH treatments throughout 4 to 5 days, which are fairly time consuming, stressful and subject to error. Furthermore, this frequent management protocol makes it nearly impossible to apply it, for example, in beef cattle kept in pastures. Therefore, the standard superovulatory approach is based on multiple injections of FSH at the time of emergence of follicular waves, which normally happens 8 to 12 days after naturally occurring estruses. This method is known as “estrus-based”, and while efficient in terms of embryo production, it is conditioned by the stage of the estrus cycle and requires estrus detection. Later studies have found that fertilization rates are significantly lower in “estrus-based” programs as compared to modern synchronization protocols that allow a more ideal interval from insemination to ovulation.

Studies have been conducted in order to determine whether lengthening the FSH stimulation protocol could increase the ovulatory response. The authors appear to conclude that extending the FSH injection period from 4 consecutive days to 7 consecutive days could sustain follicle growth and result in more follicles acquiring the capacity to ovulate. More studies are ongoing to confirm this hypothesis in different cattle breeds.

In another reported protocol, cows were treated with FSH for 3 days instead of 4, and the last 2 injections, on day 4, were replaced by 2 eCG (equine Chorionic Gonadotropin) injections (Barros et al., 2008). This treatment appeared to increase the number of embryos, although the time of treatment is still long. In addition, eCG is a fairly long molecule and has been described to induce antibody formation when repeatedly used, perhaps limiting the wide/frequent use of these types of protocols.

In the attempt to reduce the number of FSH injections, an alternative protocol has been developed wherein FSH is embedded in a slow release polymer (hyaluronan). Using this slow release formulation, a single injection was made after “estrus-based” superovulation schedules. However, the authors conclude that either the hyaluronan is too viscous or that the single intramuscular injection resulted in a lower superovulatory response (Bo et al., 2010).

EP2134165 proposes an alternative method for increasing reproduction using a single-chain FSH wherein both subunits are linked covalently by a peptide linker, which is administered in a single dose 7 to 13 days after estrus. According to this method, the animals are prepared for the FSH injection by identifying the reference estrus (or heat) date (day of last heat) and then the FSH is administered in a single injection between days 7 to 8 of the animal's cycle. The results appear satisfactory, but individual observation and monitoring of the animals is required to determine the most effective treatment regimen.

### SUMMARY OF THE INVENTION

The present invention relates to methods and compositions for increasing reproductive performance in non-human mammals using a biologically active recombinant Follicle Stimulating Hormone (rFSH). The present invention also provides improved methods for increasing reproduction, particularly fertility and fecundity, in non-human mammals.

More particularly, the invention provides improved rFSH-based compositions and treatment methods which provide improved reproductive performance in non-human mammals by minimizing the number of administrations and the dosage.

A particular object of the invention relates to an rFSH analog, or to a composition comprising an rFSH analog, for use for increasing reproductive performance in non-human mammals undergoing a follicular synchronization protocol and/or treatment. More preferably, the rFSH analog is administered in one single administration, preferably at a dose range comprised between about 0.01 μg and about 5 mg.

A particular object of the invention relates to the use of an rFSH analog, or of a composition comprising an rFSH analog, for increasing reproductive performance in non-human mammals undergoing a follicular synchronization protocol and/or treatment. More preferably, the rFSH analog is administered in one single administration, preferably at a dose range comprised between about 0.01 μg and about 5 mg.

Another object of the invention resides in an rFSH analog (or a composition comprising an rFSH analog) for use to increase reproductive performance in one or several non-human mammals, wherein said rFSH analog is administered to each of said one or several non-human mammals after follicle synchronization, in one single administration, preferably at a dose range comprised between 10 μg and 1 mg.

Another object of the invention resides in a method for increasing reproductive performance in one or several non-human mammals, comprising administering to each of said one or several non-human mammals, after follicle synchronization, one single administration of an rFSH analog, preferably at a dose range comprised between 10 μg and 1 mg.

A further object of the invention is a method for inducing superovulation in one or several non-human mammals, comprising administering to each of said one or several non-human mammals an rFSH analog, wherein said rFSH analog is administered to said non-human mammals after follicle synchronization protocol and/or treatment, in one single administration, preferably at a dose range comprised between 10 μg and 1 mg.

Another object of the invention is a method for increasing ovulation rate in one or several non-human mammals, comprising administering to each of said one or several non-human mammals an rFSH analog, wherein said rFSH analog is administered to said non-human mammals in one single administration, preferably at a dose range comprised between 10 μg and 1 mg. Administration is preferably performed after follicle synchronization.

Another object of the invention is an improved method for producing or recovering oocytes in one or several non-human mammals, comprising administering to each of said one or several non-human mammals an rFSH analog, wherein said rFSH analog is administered to said non-human mammals in one single administration, preferably at a dose range comprised between 10 μg and 1 mg.

Administration is preferably performed after follicle synchronization. Such a method is particularly advantageous for improving the performance of in vitro fertilization (IVF) protocols or procedures.

In this respect, a further object of the invention relates to an in vitro fertilization (IVF) method comprising a step of obtaining oocytes from a non-human mammal donor and a step of fertilizing said oocytes in vitro, wherein the non-human mammal donor is treated by one single administration of an rFSH analog, preferably at a dose range comprised between 10 μg and 1 mg, to increase oocyte production. Administration is preferably performed after follicle synchronization of the non-human mammal donor.

Another object of the invention is a method for improving the quality and quantity of corpora lutea in a non-human mammal, comprising administering to said non-human mammal an rFSH analog, wherein said rFSH analog is administered to said non-human mammal in one single administration, preferably at a dose range comprised between 10 μg and 1 mg.

In a particular embodiment, the invention relates to a method comprising:

(a) providing a non-human mammal (e.g., an ungulate), or a group of non-human mammals (e.g., ungulates), which has been treated to synchronize follicles, or treating a non-human mammal (e.g., an ungulate), or a group of non-human mammals (e.g., ungulates), to synchronize follicles;

(b) administering to said non-human mammal or group of mammals one single dose of an rFSH analog at a dose range comprised between about 0.01 μg and about 5 mg, preferably by injection, more preferably by intramuscular injection; and

(c) inseminating the non-human mammal or group of non-human mammals, preferably by artificial insemination, preferably near the time of ovulation, more preferably 2 to 7 days, even more preferably 4 to 6 days after administration step (b).

This method avoids the need to detect or monitor estrus, and is more effective especially for groups of non-human mammals.

In another particular embodiment, the invention relates to a method comprising:

(a) providing an ungulate or a group of ungulates which has been treated to synchronize follicles, or treating an ungulate or a group of ungulates to synchronize follicles;

(b) administering to said ungulate or group of ungulates one single dose of an rFSH analog at a dose range comprised between about 10 μg and about 1 mg, preferably by injection, more preferably by intramuscular injection;

(c) recovering oocytes from the ungulate, preferably by follicular aspiration;

(d) optionally, in vitro fertilizing the recovered oocytes; and

(e) optionally, implanting the fertilized oocytes in a recipient animal, preferably in a recipient animal previously treated by administration of one single dose of an rFSH analog at a dose range comprised between about 10 μg and about 1 mg.

The rFSH analog for use in the present invention is preferably a single chain rFSH. Furthermore, the rFSH analog is preferably used in essentially pure form, optionally in association with one or several pharmaceutically acceptable excipients or carriers.

The invention may be used in non-human mammals and preferably in any ungulate, such as bovine, ovine, equine, sheep, or goat. The compositions and method of the invention are particularly effective for increasing fertility and/or fecundity and, especially, superovulation, ovulation rate, oocyte production, or corpora lutea (CL) quality and quantity in bovines.

### LEGEND TO THE FIGURES

FIG. 1: Protocol used to increase reproductive performance with an rFSH analog.

FIG. 2: Nucleotide and amino acid sequence of a bovine rFSH analog for use in the invention. The CTP linker sequence which links the beta and alpha subunits is underlined. The underlined and bold nucleotides encode the signal peptide.

### DETAILED DESCRIPTION OF THE INVENTION

The present invention provides methods to increase reproductive performance in non-human mammals, and preferably ungulates, even more preferably cattle. In particular, a recombinant single chain FSH is used to stimulate fertility and/or fecundity in non-human female mammals, as illustrated by a superovulation, an increase of embryo production, and/or an increase of pregnancy. The recombinant single chain FSH is also effective to stimulate the growth and the maturation of follicles, resulting in an improved oocyte recovery, superovulation, and ovulation rate, and in an improved quality and quantity of corpora lutea (CL).

The recombinant single chain FSH can be used in insemination protocols and/or treatments, including for embryo transplantation and in vitro fertilization. The invention may be used with any ungulate, preferably bovine.

### DEFINITIONS

Within the context of the present invention, the term “increasing reproductive performance” or “increased reproductive performance” refers to increasing the likelihood that a non-human mammal, or a plurality of non-human mammals, will be fertile and fecund.

Increasing reproductive performance includes a stimulation of the growth and/or maturation of follicles, or an improvement in the quality and quantity of corpora lutea.

Increased reproductive performance also includes an increased likelihood that a non-human mammal, or a plurality of non-human mammals, which has been inseminated will become pregnant, deliver a live offspring, or develop viable embryos.

Increasing reproductive performance also includes increasing the number of viable embryos a non-human mammal or a plurality of non-human mammals produce in utero and/or in vitro. Increased reproductive performance includes increasing fertility, fecundity, superovulation, oocyte rate, ovulation rate, embryo production and/or pregnancies. An increase is preferably by approximately at least 1% as compared to non-treated non-human mammals, more preferably by at least 2%, 3%, 4%, 5%, 10% or more.

The term “fertility” or “fertile” refers, within the context of this invention, to the ability to produce fertilizable oocytes.

The term “fecund” or “fecundity” refers, within the context of this invention, to the ability to complete a pregnancy.

The term “superovulation” refers, within the context of this invention, to an increase in the number of ovulated follicles and/or in the creation of fertile ova.

The term “pregnant” refers to a non-human mammal or to a group of non-human mammals some of which being currently pregnant or that has been inseminated and may be pregnant.

As used herein, the term “estrus” refers to the period during which a non-human mammal is most likely to become pregnant. Estrus may be detected or monitored by behavioral demonstration that a non-human mammal is in heat, including showing standing heat.

“Insemination” refers to introducing semen by any method known in the art, including, but not limited to, natural and Artificial Insemination (AI) and in vitro fertilization (IVF).

A “group” of animals designates any group of at least 2 non-human mammals, such as a herd or flock.

An “ungulate” refers to any animal with hooves and especially the two taxonomic orders Perissodactyla and Cetartiodactyla.

The term “administration” refers to all routes of administration such as oral, enteral, mucosal, parenteral and percutaneous. Preferably the administration route is an injection.

The term “follicle synchronization” refers, within the context of this invention, to synchronization of the emergence of follicular waves.

### Recombinant FSH Analog

Follicle Stimulating Hormone belongs to the class of fertility hormones. FSH is composed of two chains (or subunits), termed alpha and beta. Under physiological conditions, both subunits are linked by non-covalent interaction. The nucleotide and amino acid sequences of FSH from distinct species have been disclosed and are available in public databases such as bovine FSH (Kim, K. E. et al., 1988, “Nucleotide sequence of the bovine gene for follicle-stimulating hormone beta-subunit”, DNA, 1988, 7(4): 227-233) or human FSH (gene bank number CAA43996.1).

The present invention utilizes an FSH analog. The term “analog” designates, generally, a compound which mimics the physiological effect of a natural compound. An analog is structurally similar to the natural compound, and may present structural differences as a result of, e.g., production methods or because the differences confer a beneficial activity to the analog.

In a preferred embodiment, an FSH analog of the invention is an FSH having both alpha and beta subunits covalently linked. Such a preferred FSH analog may also be designated “single chain” FSH.

In a preferred embodiment, the invention uses a recombinant FSH in which the alpha subunit is covalently linked to the beta subunit by a peptide linker. The invention shows that such recombinant single chain analogs of FSH provide improved properties for increasing reproductive performance in ungulates. The peptide linker may be any peptide linker which does not affect the conformation or activity of FSH. In a preferred embodiment, the linker is a CTP linker, e.g., a linker which comprises a sequence of the carboxy terminal peptide of human chorionic gonadotropin as described in U.S. Pat. No. 6,242,580 and US2008/0312151. In another embodiment, the linker is a peptide comprising the (G₄S) sequence, which may be repeated several times.

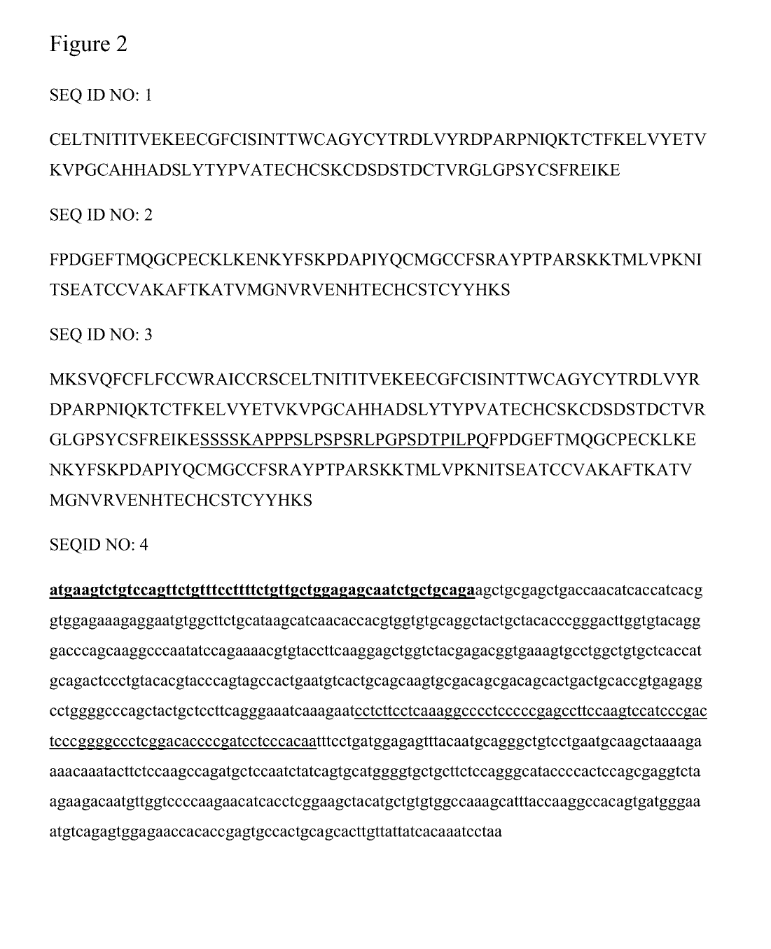

The cDNA and amino acid sequence of a recombinant bovine FSH analog for use in the present invention is provided in FIG. 2 (SEQ ID NO: 3 and SEQ ID NO: 4). The first part (amino acids 1-129) corresponds to the sequence of bovine FSH beta subunit (SEQ ID NO: 2), the central part (amino acids 130-157) corresponds to the carboxy terminal peptide linker, and the third part (amino acids 158-253) corresponds to the sequence of bovine FSH alpha subunit (SEQ ID NO: 1; see also EP2134165).

One embodiment of the invention uses a single chain recombinant bovine FSH having the amino acid sequence of SEQ ID NO: 3, or an amino acid sequence having 90% or greater, preferably 95% or greater, homology to the amino acid sequence of SEQ ID NO: 3.

When the method is for treating a bovine, it is preferred to use a bovine FSH analog as disclosed above, i.e., a recombinant FSH wherein the alpha and beta domains derive from bovine FSH and are linked by a peptide linker.

When the method is for treating another ungulate, it is preferred to use an FSH analog derived from said ungulate.

The protein is preferably essentially pure, i.e., with a purity level of at least 95%, more preferably at least 97, 98 or 99%.

In a preferred embodiment, the FSH is administered in a composition comprising a suitable pharmaceutical formulation. The pharmaceutical formulation may comprise one or several excipients or carriers.

In one preferred embodiment, the bovine FSH has an activity above 10,000 IU/mg, more preferably above 13,000 IU/mg, such as about 16,000 IU/mg. According to the conversion factor published by the ASRM practice committee (Fertility and Sterility, vol. 90, supl. 3, November 2008, American Society for Reproductive Medicine), 10,000 IU of bovine FSH corresponds to 600 μg.

### Treatment

As previously indicated, the invention relates to novel methods for increasing reproductive performance in non-human mammals using a recombinant FSH analog. The invention may be used in insemination programs, particularly ungulate artificial insemination, e.g., to improve ovulation rate and/or superovulation and/or the quality and quantity of corpora lutea in the treated animals, as well as in vitro fertilization programs, e.g., to improve oocyte recovery from donor animals and/or pregnancy rate in recipient animals.

More particularly, an object of the invention relates to a recombinant FSH analog for use to increase reproductive performance in non-human mammals, wherein said recombinant FSH comprises an alpha subunit and a beta subunit that are covalently linked by a peptide linker, and wherein said recombinant FSH is administered to said non-human mammals after follicle synchronization in one single administration, preferably at a dose comprised between 10 μg and 1 mg.

Another object of the invention resides in a method for increasing reproductive performance or embryo production in non-human mammals, comprising administering to said non-human mammals, after follicle synchronization, one single administration of a recombinant FSH analog at a dose comprised between 10 μg and 1 mg, said recombinant FSH analog comprising an alpha subunit and a beta subunit that are covalently linked by a peptide linker.

Another object of the invention resides in a method for improving oocyte recovery in non-human mammals, comprising administering to said non-human mammals, after follicle synchronization, one single administration of a recombinant FSH analog at a dose comprised between 10 μg and 1 mg, said recombinant FSH analog comprising an alpha subunit and a beta subunit that are covalently linked by a peptide linker.

Another object of the invention resides in a method for increasing pregnancy rate in non-human mammals, comprising administering to said non-human mammals, after follicle synchronization, one single administration of a recombinant FSH analog at a dose comprised between 10 μg and 1 mg, said recombinant FSH analog comprising an alpha subunit and a beta subunit that are covalently linked by a peptide linker.

Another object of the invention resides in a method for increasing ovulation rate in non-human mammals, comprising administering to non-human mammals, after follicle synchronization, one single administration of a recombinant FSH analog at a dose comprised between 10 μg and 1 mg, said recombinant FSH analog comprising an alpha subunit and a beta subunit that are covalently linked by a peptide linker.

A further object of the invention is a method for inducing superovulation in non-human mammals, comprising administering to a non-human mammal a recombinant FSH analog, wherein said recombinant FSH analog comprises an alpha subunit and a beta subunit that are covalently linked by a peptide linker, and wherein said recombinant FSH analog is administered to said mammal after follicle synchronization in one single administration, preferably at a dose comprised between 10 μg and 1 mg.

In a preferred embodiment, the method comprises:

(a) treating a non-human mammal (such as an ungulate) or a group of non-human mammals (such as a group of ungulates) to synchronize follicles, or providing a non-human mammal (such as an ungulate) or a group of non-human mammals (such as a group of ungulates) which has been treated to synchronize follicles;

(b) administering to the treated non-human mammal(s) between 10 μg and 1 mg of a recombinant FSH analog in one single dose; and

(c) optionally, inseminating the non-human mammal(s) and/or collecting oocytes preferably near the time of ovulation, more preferably 2 to 7 days, even more preferably 4 to 6 days after administration step (b).

In a first treatment step, emergence of a new follicular wave is synchronized. The invention shows that, in combination with a recombinant FSH of the invention, such a treatment allows improved embryo production, even at lower dose of hormone and with fewer injections.

Follicular growth is not continuous in non-human mammals such as bovines, but occurs in waves (2 to 4 waves per cycle). Each wave begins approximately when the dominant follicle in the previous wave gains maximum size, at which time numerous small follicles begin a period of rapid growth. From this group of follicles, one follicle is allowed to grow to a much larger size than the others. This large follicle is called the dominant follicle, because it has the ability to regulate and restrict the growth of the smaller follicles, called subordinate follicles. A few days after reaching maximum size, the dominant follicle begins to regress and die. As the dominant follicle degenerates, its ability to restrict the other follicles is reduced; therefore, a new follicular wave is initiated. A consequence of this dynamic process is that follicles of all sizes, including at least one large follicle, exist on each day of the estrous cycle.

Follicle wave synchronization gives the opportunity to treat all non-human mammals in a limited period of time and, therefore, to capture the economic benefits of the insemination. Upon synchronization of the estrous cycle, a high percentage of treated females show a fertile, closely synchronized estrus and ovulation.

The synchronization of ovulation or timed-AI protocols refers to methods and/or protocols that artificially stimulate follicle growth and timed ovulation, so that ovulation is initiated at a predetermined time with no need to monitor estrus behavior. A review of common methods and protocols applied in bovines are described in Bo et al. at the 28^{th} annual meeting AETE, Saint Malo, France, 7-8 Sep. 2012 (Recent advances in the control of follicular development and superovulation protocols in cattle).

A new follicle wave emergence can be performed by hormonal or physical treatment. Hormonal treatment comprises the administration of suitable hormones such as prostaglandin(s), progestagen(s), or GnRH. Physical treatment includes follicle ablation.

In a preferred embodiment, the follicle synchronization is obtained by hormonal treatment of the non-human mammal(s), preferably with progestagens, progestagen-prostaglandin combinations, prostaglandins alone, progestagen-estrogen combinations, and gonadotropin-prostaglandin combinations with or without progestagens.

In a preferred embodiment, follicle synchronization is obtained by one of the following treatments:
PGF2alpha or its analogs;
GnRH+PGF2alpha; or
Progestagen (as progesterone), optionally in combination with estrogen, PGF2alpha or GnRH.

Specific dosages and/or protocols are disclosed in the art such as Thatcher et al., 2001 (American Association of Bovine Practitioners (AABP), Vancouver, 95-105); Diskin et al., 2001 (occasional publication no. 26, p. 175, British Society of Animal Science); and Pursley et al., 1995 (Theriogenology, 44, p. 915). Also, progestagens may be administered using specific devices such as implants (e.g., PRID, from Ceva Santé Animale).

In another embodiment, follicle synchronization is obtained by follicular ablation. Ablation of follicles designates the elimination, removal or destruction of at least one follicle. Follicular ablation refers to physical methods of follicular ablation, such as cautery or ultrasound-guided transvaginal follicle aspiration at random stages of the estrous cycle (Bergfelt, 1997). Ablation can be directed to all follicles or only to the one or two largest follicles (Baracaldo et al., 2000), to ensure that at least the dominant follicle is suppressed.

In a preferred embodiment, the invention relates to a method comprising:

(a) treating a non-human mammal (such as an ungulate) or a group of non-human mammals (such as ungulates) with a progestagen, a progestagen-prostaglandin combination, a prostaglandin, a progestagen-estrogen combination, or a gonadotropin-prostaglandin combination with or without progestagens to synchronize follicles, or providing a non-human mammal (such as an ungulate) or a group of non-human mammals (such as ungulates) which has been treated with a progestagen, a progestagen-prostaglandin combination, a prostaglandin, a progestagen-estrogen combination, or a gonadotropin-prostaglandin combination with or without progestagens to synchronize follicles;

(b) administering to the treated non-human mammals (such as ungulates) between 10 μg, and 1 mg of a recombinant FSH analog in one single dose; and

(c) optionally, inseminating a treated non-human mammal (such as an ungulate) of (b) and/or collecting oocytes from a treated non-human mammal (such as an ungulate) of (b), preferably near the time of ovulation, more preferably 2 to 7 days, even more preferably 4 to 6 days after administration step (b).

In step (b), the recombinant FSH analog can be administered using any means or techniques known per se in the art including, without limitation, systemic administration, such as intramuscular, intravenous, subcutaneous, etc. Preferred administration route is intramuscular injection.

In a preferred embodiment, the composition or method of the invention uses a single dose of rFSH analog of 50 μg.

In another preferred embodiment, the composition or method of the invention uses a single dose of rFSH analog of 100 μg.

The results presented in the experimental section show that, on synchronized non-human mammals such as ungulates, such a single administration causes production of fertile embryos and substantially increases the reproductive activity.

In a preferred embodiment, the method further comprises a step (c) of inseminating said ungulate, preferably near the time of ovulation, more preferably 2 to 7 days, even more preferably 4 to 6 days after rFSH analog administration.

Additional hormones, such as luteinizing hormone, chorionic gonadotropin and prostaglandin, are optionally administered as well as the rFSH. In one embodiment, prostaglandin is administered to the non-human mammal in addition to administration of the rFSH analog. The prostaglandin is optionally administered as a single dose, typically by injection, or as multiple doses administered several hours apart. In one embodiment, a first dose of prostaglandin is given to the non-human mammal after administration of the rFSH analog followed by a second dose of prostaglandin which is given to the non-human mammal approximately 6 hours to 1 day following the first prostaglandin dose.

Alternatively, the method comprises a step (c) of recovering or collecting oocytes from a treated non-human mammal of (b), preferably near the time of ovulation, more preferably 2 to 7 days, even more preferably 4 to 6 days after rFSH analog administration. Oocytes may be recovered by techniques known per se in the art such as, without limitation, follicular aspiration. The collected oocytes display improved characteristics for fecundity. The collected oocytes may be fertilized in vitro, and subsequently transferred to recipient non-human mammals, e.g., ungulates.

The invention may be used in any ungulate, such as bovines, sheep, goats, cervids, yaks, water buffalo, bison, antelopes, gazelles, elk, reindeer, moose, bighorn sheep, giraffes, camelids, swine, equines, alpacas, and vicunas. It is particularly suited for treating female beef and dairy cattle, including heifers.

Further aspects and advantages of the invention will be disclosed in the following experimental section, which illustrates the claimed invention.

### EXAMPLES

### Example 1—Effect on Reproduction Performance of a Single 50 μg rFSH Analog Administration in Synchronized Non-Human Mammals

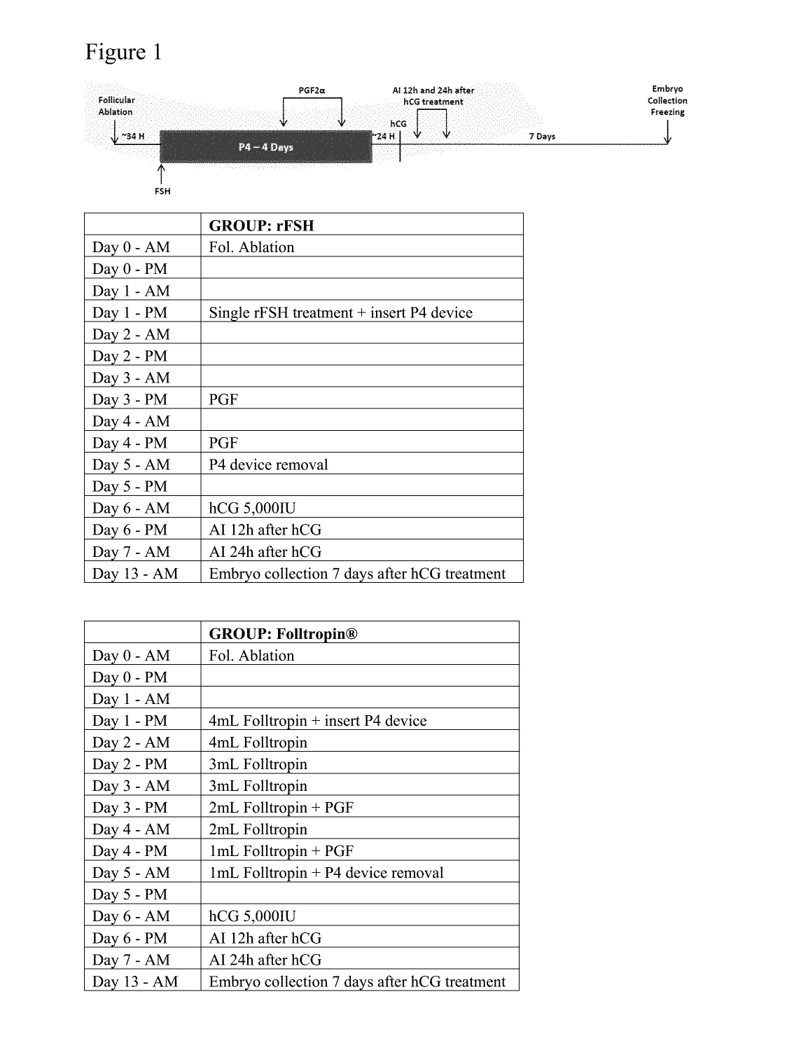

We treated 14 dairy heifers with one single intramuscular injection of bovine rFSH analog (50 μg/heifer). Injection was performed 30 h (in a 24 to 48 h window) after follicular ablation. The bovine rFSH analog used is as depicted in FIG. 2. The treatment protocol is represented in FIG. 1.

All groups received two injections of prostaglandin PGF2α at 2 days and 3 days after the first FSH injection (F0). hCG (human Chorionic Gonadotropin) injection was performed at 5 days after F0 and Artificial Insemination (AI) was performed 12 h and 24 h after the hCG treatment. Embryo collection by non-surgical procedure occurred 7 days after AI.

Folltropin®-V (Bioniche Animal Health Product, Canada) was used a reference treatment. Folltropin®-V is a purified lyophilized follitropin extract obtained from porcine pituitary glands. Folltropin®-V (50 mg) was administered intramuscularly, twice daily, in decreasing doses for four days (cumulated FSH at the end of treatment is 400 mg/heifer). 14 dairy heifers were treated with Folltropin®-V.

Different parameters were measured:
Superovulation: superovulation was considered successful if >2CL (corpora lutea) on the day of the flush;
Ovulation rate: based on overall group LSM (Least Squares Means) of ovulatory response per cow; and
Embryo quality: based on overall group LSM (Least Squares Means) of number of viable embryos per cow.

The results are presented in the following table.

| | rFSH/single injection |
|---|---|
| | 50 μg |
| Heifers enrolled (n) | 14 |
| Heifers superovulated (n)* | 13 |
| Heifers superovulated (%) | 92.8 |
| Ovulation rate | 81% |
| Average number of CL | 14.2 |
| Number of viable embryos (VE) | 7.9 |
| VE vs. Folltropin ®-V VE | 134% |

The results show that the treatment of the invention can clearly induce superovulation response in cattle. As a comparison, in the heifers treated with the reference protocol (8 injections of a natural FSH such as Folltropin®-V, 400 mg), the treatment of the invention (1 single injection of an rFSH analog, 50 μg) resulted in a higher ovulation rate (81% vs 76%) and a higher number of viable embryos (7.9 vs 5.9). Accordingly, even if one heifer did not superovulate with the treatment of the invention (while all did with the reference treatment), the quality of the embryos seems better with the invention. Accordingly, the combination of a follicle synchronization treatment with a single rFSH analog injection allows consistent production of superovulated embryos.

### Example 2—Effect on Reproduction Performance of a Single 100 μg rFSH Analog Administration in Synchronized Non-Human Mammals

We treated 14 dairy heifers with one single intramuscular injection of bovine rFSH analog (100 μg/heifer). Injection was performed 30 h (in a 24 to 48 h window) after follicular ablation. The bovine rFSH analog used is as depicted in FIG. 2. The treatment protocol is represented in FIG. 1.

All groups received two injections of prostaglandin PGF2α at 2 days and 3 days after the first FSH injection (F0). hCG (human Chorionic Gonadotropin) injection was performed at 5 days after the F0 and Artificial Insemination (AI) was performed 12 h and 24 h after the hCG treatment. Embryo collection by non-surgical procedure occurred 7 days after AI.

Folltropin®-V (Bioniche Animal Health Product, Canada) was used a reference treatment. Folltropin®-V is a purified lyophilized follitropin extract obtained from porcine pituitary glands. Folltropin®-V (50 mg) was administered intramuscularly, twice daily, in decreasing doses for four days (cumulated FSH at the end of treatment is 400 mg/heifer). 14 dairy heifers were treated with Folltropin®-V.

Different parameters were measured:
Superovulation: superovulation was considered successful if >2CL (corpora lutea) on the day of the flush;
Ovulation rate: based on overall group LSM (Least Squares Means) of ovulatory response per cow; and
Embryo quality: based on overall group LSM (Least Squares Means) of number of viable embryos per cow.

The results are presented in the following table.

| | rFSH/single injection |
|---|---|
| | 100 μg |
| Heifers enrolled (n) | 14 |
| Heifers superovulated (n)* | 12 |
| Heifers superovulated (%) | 85.7 |
| Ovulation rate | 88% |
| Average number of CL | 14.7 |
| Number of viable embryos (VE) | 3.8 |

The results show that the treatment of the invention can clearly induce a superovulation response in cattle. As a comparison, in the heifers treated with the reference protocol (8 injections of a natural FSH such as Folltropin®-V, 400 mg), the treatment of the invention (1 single injection of a rFSH analog, 100 μg) resulted in a higher ovulation rate (88% vs 76%). Accordingly, even if two heifers did not superovulate with the treatment of the invention (while all did with the reference treatment), the quality of the embryos seems equivalent between the two products. The treatment achieved more than 70% of superovulatory response and also met the criteria in terms of embryo quality. Accordingly, the combination of a follicle synchronization treatment with a single rFSH analog injection allows consistent production of superovulated embryos.

We claim:

1. A method for increasing reproductive performance in a non-human mammal, comprising administering to said non-human mammal a recombinant FSH (rFSH) analog, or a composition comprising an rFSH analog, wherein said rFSH analog is administered to said non-human mammal after follicle synchronization and in one single administration.

2. The method of claim 1, wherein the rFSH is administered in one single administration at a dose range between about 0.01 μg and about 5 mg.

3. The method of claim 2, wherein the rFSH is administered in one single administration at a dose range between about 1 μg and 2 mg, or between about 10 μg and 1 mg.

4. The method of claim 3, wherein the rFSH is administered in one single administration at a dose of 50 μg or 100 μg.

5. The method of claim 1, wherein said rFSH is administered by systemic administration.

6. The method of claim 1, wherein said rFSH analog comprises an alpha subunit having a sequence of a non-human mammal FSH alpha chain, and a beta subunit having a sequence of a non-human mammal FSH beta chain of the same species as the alpha chain, and wherein said alpha and beta subunits are covalently linked by a peptide linker.

7. The method of claim 6, wherein the rFSH analog is an ungulate recombinant FSH analog.

8. The method of claim 7, wherein the alpha subunit is a polypeptide comprising a sequence that is at least 95% homologous to SEQ ID NO 1 and the beta subunit is a polypeptide comprising a sequence that is at least 95% homologous to SEQ ID NO 2.

9. The method of claim 1, wherein the non-human mammal is bovine, sheep, goat, cervid, yak, water buffalo, bison, antelope, gazelle, elk, reindeer, moose, bighorn sheep, giraffe, camelid, swine, equine, alpaca or vicuna.

10. The method of claim 9, wherein the non-human mammal is a vicuna.

11. The method of claim 1, wherein the rFSH comprises an amino acid sequence having at least 95% homology with SEQ ID NO 3.

12. The method of claim 9, wherein the non-human mammal is a bovine.

13. The method of claim 1, further comprising collecting oocytes from said non-human mammal near the time of ovulation and after rFSH analog administration.

14. The method of claim 1, wherein said rFSH is injected in combination with at least one prostaglandin.

15. The method of claim 1, wherein said rFSH is injected in combination with a progestagen based treatment.

16. The method of claim 1, comprising:
(a) providing a non-human mammal or a group of non-human mammals which has been treated to synchronize follicles;
(b) administering to said non-human mammal or group of non-human mammals one single dose of an rFSH analog at a dose range between about 0.01 μg and about 5 mg; and
(c) optionally, inseminating the non-human mammal or group of non-human mammals or collecting oocytes from the non-human mammal or group of non-human mammals near the time of ovulation and after administration step (b).

17. The method of claim 1, comprising:
(a) treating a non-human mammal or a group of non-human mammals to synchronize follicles;
(b) administering to said non-human mammal or group of non-human mammals one single dose of an rFSH analog at a dose range between about 0.01 μg and about 5 mg; and
(c) optionally, inseminating the non-human mammal or group of non-human mammals by artificial insemination or collecting oocytes from the non-human mammal or group of non-human mammals near the time of ovulation and after administration step (b).

18. The method of claim 16, wherein follicle synchronization is obtained by hormonal treatment of the non-human mammal with a prostaglandin, a progestagen, and/or GnRH.

19. The method of claim 18, wherein follicle synchronization is obtained by a combined administration of a progestagen and prostaglandin PGF2alpha.

20. A method to increase reproductive performance in a bovine or group of bovine, wherein the method comprises:
(a) administering to the bovine, or to at least two members of the group of bovine, a prostaglandin, progestagen, and/or GnRH in an amount effective to induce follicle synchronization; and
(b) administering to the follicle-synchronized bovine, in one single administration, between 10 μg and 1 mg of a recombinant FSH analog.


SEQUENCE LISTING (4 sequences)

SEQ ID NO: 1 - protein, 109 aa, Bos taurus
  MISC_FEATURE (1)..(109): bovine FSH alpha subunit
CELTNITITVEKEECGFCISINTTWCAGYCYTRDLVYRDPARPNIQKTCTFKELVYETVKVPGCAHHADSLYTYPVATECHCSKCDSDSTDCTVRGLGPSYCSFREIKE

SEQ ID NO: 2 - protein, 96 aa, Bos taurus
  MISC_FEATURE (1)..(96): bovine FSH beta subunit
FPDGEFTMQGCPECKLKENKYFSKPDAPIYQCMGCCFSRAYPTPARSKKTMLVPKNITSEATCCVAKAFTKATVMGNVRVENHTECHCSTCYYHKS

SEQ ID NO: 3 - protein, 253 aa, Artificial Sequence
  Bovine rFSH analog
  MISC_FEATURE (1)..(129): bovine FSH beta subunit
  MISC_FEATURE (130)..(157): carboxy terminal peptide linker
  MISC_FEATURE (158)..(253): bovine FSH alpha subunit
MKSVQFCFLFCCWRAICCRSCELTNITITVEKEECGFCISINTTWCAGYCYTRDLVYRDPARPNIQKTCTFKELVYETVKVPGCAHHADSLYTYPVATECHCSKCDSDSTDCTVRGLGPSYCSFREIKESSSSKAPPPSLPSPSRLPGPSDTPILPQFPDGEFTMQGCPECKLKENKYFSKPDAPIYQCMGCCFSRAYPTPARSKKTMLVPKNITSEATCCVAKAFTKATVMGNVRVENHTECHCSTCYYHKS

SEQ ID NO: 4 - DNA, 762 bp, Artificial Sequence
  Bovine rFSH analog
  misc_feature (1)..(387): bovine FSH beta subunit
  misc_feature (388)..(471): carboxy terminal peptide linker
  misc_feature (472)..(762): bovine FSH alpha subunit
atgaagtctgtccagttctgtttccttttctgttgctggagagcaatctgctgcagaagctgcgagctgaccaacatcaccatcacggtggagaaagaggaatgtggcttctgcataagcatcaacaccacgtggtgtgcaggctactgctacacccgggacttggtgtacagggacccagcaaggcccaatatccagaaaacgtgtaccttcaaggagctggtctacgagacggtgaaagtgcctggctgtgctcaccatgcagactccctgtacacgtacccagtagccactgaatgtcactgcagcaagtgcgacagcgacagcactgactgcaccgtgagaggcctggggcccagctactgctccttcagggaaatcaaagaatcctcttcctcaaaggcccctcccccgagccttccaagtccatcccgactcccggggccctcggacaccccgatcctcccacaatttcctgatggagagtttacaatgcagggctgtcctgaatgcaagctaaaagaaaacaaatacttctccaagccagatgctccaatctatcagtgcatggggtgctgcttctccagggcataccccactccagcgaggtctaagaagacaatgttggtccccaagaacatcacctcggaagctacatgctgtgtggccaaagcatttaccaaggccacagtgatgggaaatgtcagagtggagaaccacaccgagtgccactgcagcacttgttattatcacaaatcctaa